(19) United States
(12) Patent Application Publication
(10) Pub. No.: US 2017/0274148 A1
(43) Pub. Date: Sep. 28, 2017

(54) DRUG DELIVERY DEVICE WITH COMBINED SETTING AND RELEASE MEMBER

(71) Applicant: Novo Nordisk A/S, Bagsvaerd (DK)

(72) Inventors: Steffen Mews, Rostock (DE); Brian Jensen, Broenshoej (DK)

(73) Assignee: Novo Nordisk A/S, Bagsvaerd (DK)

(21) Appl. No.: 15/529,287

(22) PCT Filed: Dec. 15, 2015
(86) PCT No.: PCT/EP2015/079862

§ 371 Date: May 24, 2017

(30) Foreign Application Priority Data
Dec. 16, 2014 (EP) 14198354.4
Feb. 3, 2015 (EP) 15153603.4

(51) Int. Cl. A61M 5/315; A61M 5/24; A61M 5/20

(52) U.S. Cl. CPC A61M 5/31553; A61M 5/2033; A61M 5/2422; A61M 5/3158; A61M 5/31593; A61M 2005/2407; A61M 2005/2411; A61M 2005/3126

(57) ABSTRACT

Drug delivery device has drive assembly adapted to move a piston rod in a distal direction, the drive assembly comprising a drive spring. A rotatable setting member allows a user to set a dose amount to be expelled, the drive spring being strained during dose setting. User actuated release means is provided for releasing the drive spring to thereby move the piston rod in the distal direction corresponding to the set dose. The setting member can be moved axially between an initial position and a proximal setting position, and between the initial position and a distal expelling position, wherein the setting member cannot be rotated to set a dose when in the initial position, the setting member can be rotated to set a dose when in the proximal position, and a strained drive spring is released when the setting member is moved axially from the initial to the distal position.

The present invention generally relates to drug delivery devices adapted to be used and operated by a patient on his or her own hand. In specific embodiments the invention relates to medical delivery devices of the spring-driven type.

### BACKGROUND OF THE INVENTION

In the disclosure of the present invention reference is mostly made to the treatment of diabetes by delivery of insulin or other diabetes drugs, however, this is only an exemplary use of the present invention.

Drug delivery devices in the form of injection devices for subcutaneous administration of fluid drugs have greatly improved the lives of patients who must self-administer drugs and biological agents. Drug injection devices may take many forms, including simple disposable devices that are little more than an ampoule with an injection means or they may be highly sophisticated electronically controlled instruments with numerous functions. Regardless of their form, they have proven to be great aids in assisting patients to self-administer injectable drugs and biological agents. They also greatly assist care givers in administering injectable medicines to those incapable of performing self-injections.

In particular pen-style injection devices have proven to provide an accurate, convenient, and often discrete, way to administer drugs and biological agents, such as insulin. Typically, injection devices use a pre-filled cartridge containing the medication of interest, e.g. 1.5 or 3.0 ml of insulin or growth hormone. The cartridge is typically in the form of a generally cylindrical transparent ampoule with a needle pierceable septum at one end and an opposed piston designed to be moved by the dosing mechanism of the injection device. The injection devices generally are of two types: “Durable” devices and “disposable” devices. A durable device is designed to allow a user to replace one cartridge with another cartridge, typically a new cartridge in place of an empty cartridge. In contrast, a disposable device is provided with an integrated cartridge which cannot be replaced by the user; when the cartridge is empty the entire device is discarded.

A further distinction can be made for the drive means delivering the force to move the cartridge piston forwards during expelling of a dose of drug. Traditionally injection devices have been manually actuated by the user pushing an extendable button during expelling, however, alternatively the driving force may be provided by a spring being pre-strained or strained during dose setting and subsequently released, this allowing for “automatic” dispensing of drug.

Although some injection devices are designed for delivery of a fixed dose, either a single fixed dose corresponding to the amount of drug in the cartridge or a number of fixed doses from a larger cartridge, the majority of injection devices comprises dose setting means allowing a user to set a desired size for the dose of drug to be expelled.

WO 2011/142598 and WO 2014/170267 disclose pen devices with a rotatable dose setting knob and a release button which will lock the knob when depressed. US 2011/0092905 discloses a pre-wind autopen (i.e. the spring is not strained during dose setting) where the dose setting knob has to be moved proximally to set a dose. When the knob is moved back to the initial rotationally locked position the set dose is expelled.

Having regard to the above, it is an object of the present invention to provide a drug delivery device of the spring-driven type which is both safe and user-friendly. The safety and user-oriented features should be intuitive and easy to understand and should be provided in a cost-effective manner.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in accordance with a first aspect of the invention a drug delivery device is provided comprising or being adapted to receive a cartridge with an axially displaceable piston, the drug delivery device comprising, a housing comprising a distal end and a proximal end, a piston rod adapted to engage and axially displace a piston in a loaded cartridge in a distal direction to thereby expel a dose of drug from the cartridge, and drive means adapted to move the piston rod in the distal direction and comprising a drive spring. The drug delivery device further comprises a rotatable setting member allowing a user to set a dose amount to be expelted, the drive spring being strained during dose setting or being pre-strained, and user actuated release means for releasing the drive spring to thereby move the piston rod in the distal direction corresponding to the set dose. The setting member can be moved axially between an initial position and a proximal setting position, and between the initial position and a distal expelling position. When in the initial position the setting member cannot be rotated to set a dose, whereas when in the proximal position the setting member can be rotated to set a dose. When the setting member is moved axially from the initial position to the distal position a strained drive spring is released.

By this arrangement it is prevented to a high degree that a dose is inadvertently set and release, e.g. when a traditional spring-driven device is carried in a bag or in a pocket. Further, as the setting member serves as a combined setting and release member a simple design can be achieved.

In an exemplary embodiment the setting member is disabled in the initial position by being rotationally locked. In this way a set dose can be “parked” when the setting member is moved to the initial position after a dose has been set.

The expelling mechanism may be adapted such that expelling of a set dose amount can be paused when the setting member is moved axially from the distal position to the initial position. In this way the user is allowed to control drug flow during the expelling event. The setting member may be moved axially from the initial position to the distal position against a biasing force adapted to return the setting member to the initial position.

The drug delivery device may further comprise a sensor system adapted to detect rotational movement during dose setting and dose expelling, the system comprising electronic circuitry and a switch which is in a first state when the setting member is in an expelling position, i.e. the initial or the distal position, and a second state when the setting member is in the proximal setting position, whereby a set dose is detected when the setting member is in the proximal position and an expelled dose is detected when the setting member is in the expelling position.

The sensor system may be provided with an electronically controlled display adapted to display information relating to an expelled dose of drug. The display may be arranged corresponding to the proximal end surface 389 and may be turned on e.g. when the setting member is moved from the initial to the proximal position or the display may be turned on when the dose dial is turned away from and then back to zero. Alternatively or in addition the display may be turned on when the setting member is moved from the distal to the initial position and it has been detected that a set dose has been expelled. In an exemplary embodiment the display is turned on when a set dose has been fully expelled. To save energy the display may turn off after a pre-determined amount of time, e.g. 5, 10 or 30 seconds.

The electronic circuitry may be coupled to the setting member and move axially therewith. For example, the setting member may be provided with an interior space in which at least a portion of the electronic circuitry is arranged.

In an exemplary embodiment the drug delivery device comprises a ratchet mechanism and an input member. The ratchet mechanism comprises a first ratchet part being stationary during dose setting and a second part being rotatable relative to the first part during dose setting, as well as bias means, e.g. a spring, urging the first and second ratchet parts into engagement. The second ratchet part may be coupled to the setting member or it may be formed integrally therewith. The input member is arranged to be rotated during dose setting corresponding to a set dose, e.g. clockwise during dose setting (“dial up”) and counterclockwise during dose adjusting (“dial down”) as seen from the proximal end of the device.

The drug delivery device may be provided with a drive spring connected, directly or indirectly, to the input member, such that the drive spring is strained corresponding to the dose being set, the drug delivery device further being provided with user actuatable release means for releasing the strained drive spring. In such an arrangement the ratchet mechanism is adapted to hold the input member in a rotational position corresponding to a set dose amount against the force of the strained drive spring. In the above-described embodiments the release means is in the form of the setting member.

The above-described drug delivery devices may be provided with a drug reservoir, either in the form of a prefilled device or in the form of a durable device being loaded with a thereto corresponding drug cartridge, comprising a fluid insulin drug formulation.

In a further aspect of the invention a drug delivery system is provided, the system comprising a drug delivery assembly, a first setting member comprising coupling means and electronic circuitry, and a second setting member comprising coupling means and no electronic circuitry. The drug delivery assembly comprises a housing comprising a distal end and a proximal end, a piston rod adapted to engage and axially displace a piston in a loaded cartridge in a distal direction to thereby expel a dose of drug from the cartridge, an expelling mechanism adapted to move the piston rod in the distal direction and comprising a drive spring, the expelling mechanism being adapted to allow a user to (i) set a dose amount to be expelled, the drive spring being strained during dose setting or being pre-strained, and (ii) release the drive spring to thereby move the piston rod in the distal direction corresponding to the set dose, and coupling means allowing either of the first and second setting members to be attached to the drug delivery assembly to form a drug delivery device. When a setting member is attached the setting member can be moved axially between an initial position and a proximal setting position, and between the initial position and a distal position. In the initial position the setting member cannot be rotated to set a dose, when in the proximal position the setting member can be rotated to set a dose, and when the setting member is moved axially from the initial position to the distal position a strained drive spring is released.

In an exemplary embodiment the electronic circuitry of the first setting member provides a sensor system adapted to detect rotational movement during dose setting and dose expelling. The sensor system may be provided with a switch which is in a first state when the setting member is in the initial position, a second state when the setting member in the proximal position, and the first state when the setting member is in the distal position, whereby a set dose is detected when the setting member is in the proximal position and an expelled dose is detected when the setting member is in the distal position.

As used herein, the term “drug” is meant to encompass any drug-containing flowable medicine capable of being passed through a delivery means such as a cannula or hollow needle in a controlled manner, such as a liquid, solution, gel or fine suspension. Representative drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid (dispensed) or liquid form. In the description of the exemplary embodiments reference will be made to the use of insulin containing drugs. Correspondingly, the term “subcutaneous” infusion is meant to encompass any method of transcutaneous delivery to a subject.

### BRIEF DESCRIPTION OF THE DRAWINGS

In the following the invention will be further described with reference to the drawings, wherein

FIGS. 1A and 1B show an embodiment of a drug delivery device,

FIGS. 2A and 2B show an embodiment of a further drug delivery device,

FIGS. 3A-3C show in cross-section a proximal portion of an embodiment of a drug delivery device in different operational states, and

FIGS. 4A-4F show different user-related operational states for a drug delivery device.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

Before turning to an embodiment of the present invention per se, examples of “generic” spring-driven drug delivery devices will be described, such devices providing a basis for the exemplary embodiment of the present invention.

The pen device 100 comprises a cap part 107 and a main part having a proximal body or drive assembly portion with a housing 101 in which a drug expelling mechanism is arranged or integrated, and a distal cartridge holder portion 110 in which a drug-filled transparent cartridge 120 with a distal needle-penetrable septum is arranged and retained in place by a cartridge holder attached to the proximal portion, the cartridge holder having openings allowing a portion of the cartridge to be inspected. Distal coupling means 111 allows a needle assembly to be releasably mounted in fluid communication with the cartridge interior. The cartridge is provided with a piston driven by a piston rod forming part of the expelling mechanism and may for example contain an insulin, GLP-1 or growth hormone formulation. The expelling mechanism comprises a drive spring which can be strained during dose setting and then subsequently released to drive the piston rod. A proximal-most rotatable dose setting member 180 serves to manually set a desired dose of drug shown in display window or opening 102 and at the same time strain the drive spring correspondingly, which then can be released and the dose expelled when the release button 190 is actuated.

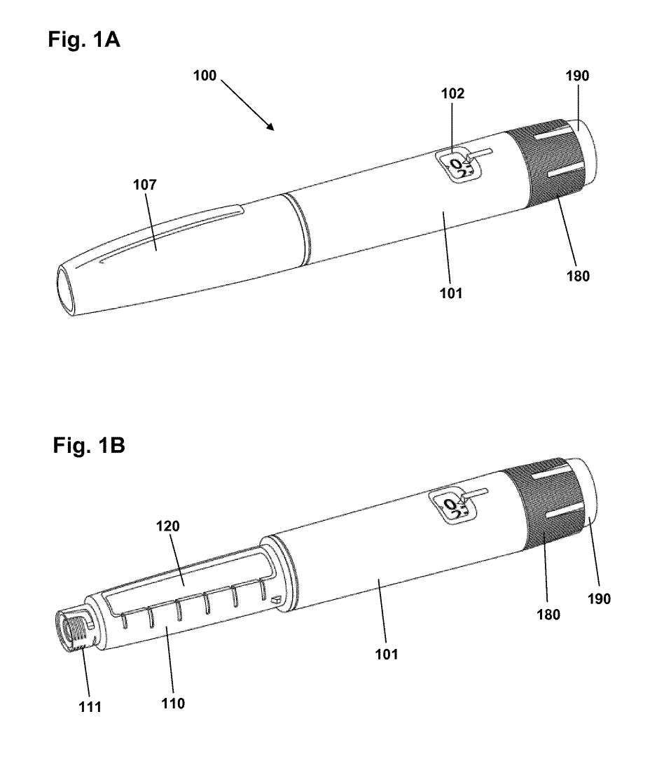

FIGS. 1A and 1B show a drug delivery device of the pre-filled type, i.e. it is supplied with a pre-mounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

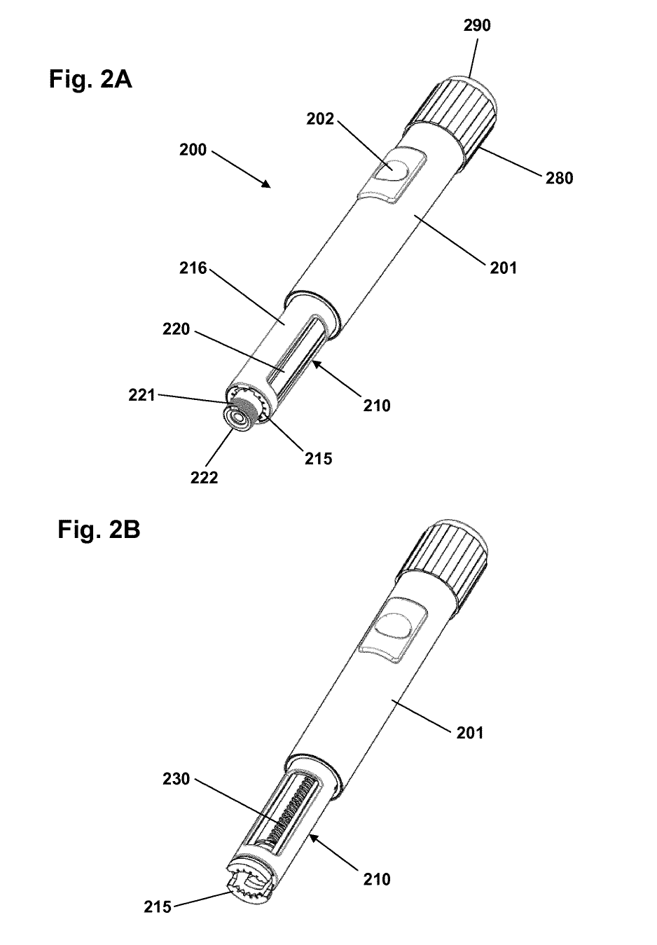

Correspondingly, with reference to FIGS. 2A and 2B a “generic” front-loaded drug delivery device will be described. More specifically, the pen device 200 comprises a cap part (not shown) and a main part having a proximal body or drive assembly portion with a housing 201 in which a drug expelling mechanism is arranged or integrated, and a distal cartridge holder portion in which a drug-filled transparent cartridge 220 with a distal needle-penetrable septum can be arranged and retained in place by a cartridge holder 210 attached to the proximal portion, the cartridge holder having openings allowing a portion of the cartridge to be inspected. The cartridge may for example contain an insulin, GLP-1 or growth hormone formulation. The cartridge is provided with distal coupling means in the form of a needle hub mount 222 having, in the shown example, an external thread 221 adapted to engage an inner thread of a corresponding hub of a needle assembly. In alternative embodiments the thread may be combined with or replaced by other connection means, e.g. a bayonet coupling. The device is designed to be loaded by the user with a new cartridge through a distal receiving opening in the cartridge holder, the cartridge being provided with a piston driven by a piston rod 230 forming part of the expelling mechanism. A proximal-most rotatable dose ring member 280 serves to manually set a desired dose of drug shown in display window 202 and which can then be expelled when the release button 290 is actuated. Depending on the type of expelling mechanism embodied in the drug delivery device, the expelling mechanism may comprise a spring which is strained during dose setting and then released to drive the piston rod when the release button is actuated. Alternatively the spring may be pre-strained such that the stored energy is released in “portions” corresponding to a set dose, e.g. as disclosed in WO 2010/070038 and WO 2014/166887 which are hereby incorporated by reference.

The cartridge holder comprises a distal opening adapted to receive a cartridge. More specifically, the cartridge holder comprises an outer rotatable tube member 216 operated by the user to control movement of gripping means to thereby open and close gripping shoulders 215 configured to grip and hold a cartridge. FIG. 2B shows the device with the cartridge removed and the gripping shoulders in their un-locked “open” position in which a cartridge can be removed and a new inserted.

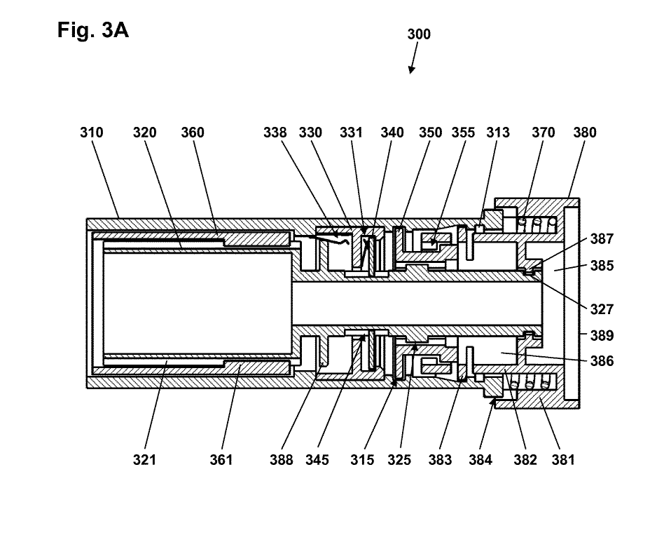

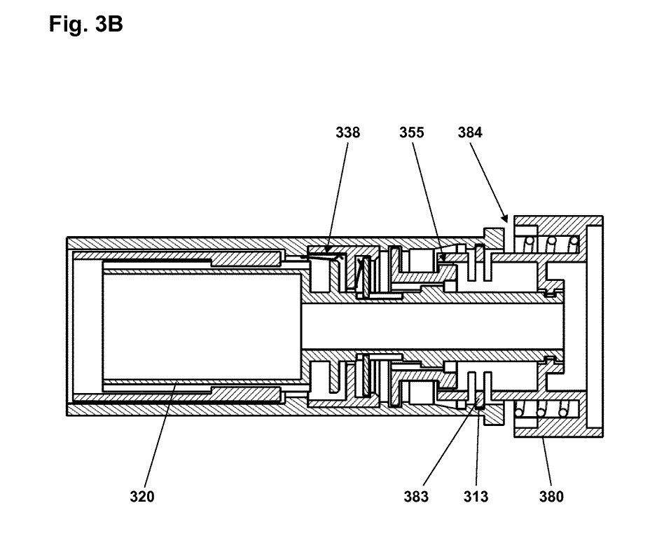

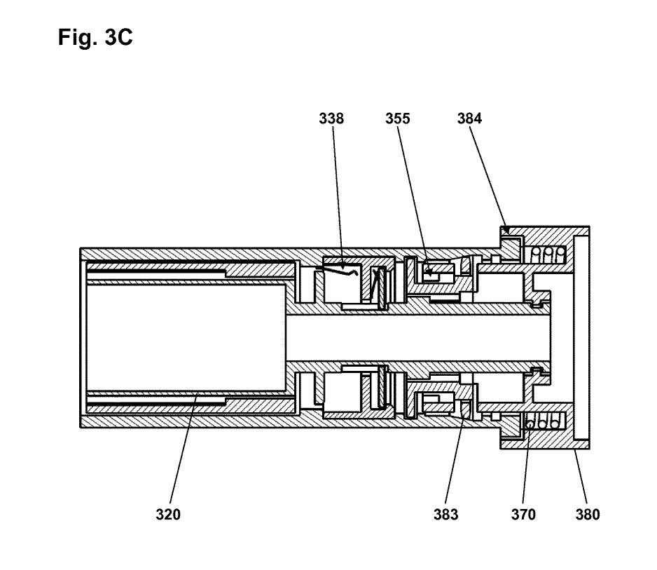

FIGS. 3A-3C show in cross-sectional views the proximal portion of a pen-formed drug delivery device in different operational states or modes: (i) an initial or rest mode in FIG. 3A, (ii) a dial mode in which a dose to be expelled can be set in FIG. 3B, and (iii) a dosing mode in which the set dose is expelled in FIG. 3C. Mainly the components relevant for illustrating the concept of the present invention are shown. More specifically, the figures show in detail the dose setting dial and release means adapted to work with a “generic” spring driven expelling mechanism, the mechanism being represented by an input member in the form of a combined coupling and drive member on which a strained spring (not shown) acts during both dose setting and when released during dose expelling. The term “drive and coupling member” indicates that this member is the member which during expelling mode drives the remaining expelling mechanism and which, when moved axially, also serves to actuate the couplings controlling the expelling action.

Examples of such expelling mechanisms can be found in e.g. WO 2014/161952 disclosing an expelling mechanism comprising a helical torque spring, and U.S. Pat. No. 8,048,037 disclosing an expelling mechanism comprising a clock-type spiral torque spring. It should be emphasized that the dose setting dial and release means in most cases should be considered part of a complete expelling mechanism as in most designs it is not possible or meaningful to separate the dose setting and dose expelling parts of the mechanism as they structurally and functionally are formed as an integral mechanism. In the shown embodiment an optional switch and rotary sensor arrangement is incorporated.

Turning to FIG. 3A the shown embodiment comprises a tubular housing 310, a combined drive and coupling member 320 serving as an input member and having a smaller-diameter proximal portion 321, a switch and sensor assembly comprising a stationary portion 330 and a rotatable portion 340, a generally tubular transition member 350, a scale drum 360 with an outer helically arranged row of dose numerals, a proximal bias spring 370, and a combined dose setting and release member (in the following dial member) 380 comprising an outer cylindrical portion 381 adapted to be gripped by the user, and an inner generally cylindrical skirt portion 382. In the interior of the dial member two cavities 385, 386 are provided which may be in communication with each other and which may be used to house electronic circuitry as described below. The proximal end surface 389 of the dial member provides a push surface for the user.

The dial member 380 is coupled axially locked but rotationally free to the proximal end of the drive and coupling member 320 via an inner circumferential ridge 387 engaging a circumferential groove 327. Between the dial member and the housing proximal end a releasable spline rotary lock 384 is provided. The housing proximal end inner surface is further provided with a circumferential locking groove 313 adapted to releasably engage a number of flexible radial extensions 383 arranged on the dial member skirt portion, to thereby provide a releasable axial parking lock. The transition member 350 is coupled axially locked but rotationally free to the housing inner surface via a ridge and groove connection 315, the coupling and drive tube 320 being guided rotationally locked but axially free in the transition member by a spline connection 325. A releasable splined dosing coupling 355 is provided between the dial member distal end and the transition member, the dosing coupling having an engaged mode in which the dial member and the coupling and drive tube are rotationally locked to each other, as well as a disengaged mode in which the two members are allowed to rotate relative to each other. The scale drum is on the inside connected rotationally locked but axially free to the coupling and drive member 320 by a spline connection 321, 361. The outside of the scale drum is in threaded helical engagement with the housing inner surface such that the scale drum is moved axially during dose setting, whereby a numeral corresponding to the currently set dose size is shown in the housing display window.

The switch and sensor assembly stationary portion 330 is locked to the housing inner surface with the rotatable sensor portion 340 being axially locked thereto but rotationally free. The rotatable switch portion is connected rotationally locked but axially free to the coupling and drive member 320 by a spline connection 345. A rotary sensor 331 is formed between the stationary and rotatable portions, and an axial switch 338 is formed between the stationary portion and the coupling and drive tube, the latter comprising a circumferential flange 388 for actuating the axial switch between an off-state and an on-state (see below).

Not part of the present invention and corresponding to the above-described generic spring-driven devices, the shown embodiment further comprises a ratchet coupling, a piston rod, a rotatable piston driver, as well as a drive coupling arranged between the drive and coupling tube and the piston driver. The ratchet coupling has an engaged state in which the coupling and drive tube can be rotated bi-directionally straining the drive spring, and a disengaged state in which the strained spring is allowed to rotate the coupling and drive tube. The drive coupling has a disengaged state in which the drive and coupling tube can be rotated relative to the piston driver, and an engaged state in which the drive and coupling tube and the piston driver will rotate together. Examples of ratchet couplings are described in greater detail in EP application 15156962 which is hereby incorporated by reference.

As indicated above FIG. 3A shows the drug delivery device in the rest mode. The rest mode is characterized by the rotary lock 384 being in a locked state, the parking lock 313, 383 being in the distal position, the drive coupling being in the disengaged state, the ratchet coupling being in the engaged state, the dosing coupling 355 being in the disengaged state, and the axial switch being in the off-state.

FIG. 3B shows the drug delivery device in the dial mode in which the dial member 380 and thereby the coupling and drive member 320 have been moved proximally. The dial mode is characterized by the rotary lock 384 being in an un-locked state allowing the dial member 380 to rotate relative to the housing, the parking lock 313, 383 being in the proximal position, the drive coupling being in the disengaged state, the ratchet coupling being in the engaged state, the dosing coupling 355 being in the engaged state, and the axial switch 338 being in the on-state. As appears, in the dial mode the user can set a dose and correspondingly strain the drive spring.

Finally, FIG. 3C shows the drug delivery device in the dosing mode in which the dial member 380 and thereby the coupling and drive member 320 have been moved to a distal-most position by the user pushing the dial member distally. Before reaching the dosing position the dial member passes through the rest mode position with the different structures in the above-described state apart from the drive spring if a dose has been set. The dosing mode is characterized by the rotary lock 384 being in a locked state, the parking lock 313, 383 being in a further-distal position, the drive coupling being in the engaged state, the ratchet coupling being in the disengaged state, the dosing coupling 355 being in the disengaged state, and the axial switch 338 being in the off-state. As appears, it is important that the ratchet coupling does not disengage before the drive coupling has engaged. Further, the proximal bias spring is compressed. In addition, a further bias spring associated with the drive and/or ratchet coupling may act on the coupling and drive tube and thus on the dial member.

When the user releases the pressure on the dial member the dial member and the other components return to the positions and states corresponding to the rest mode. If the set dose has not been fully expelled the coupling and drive tube will be in a rotational position corresponding to the remaining non-expelled dose. The user may choose to actuate the expelling mechanism again or reset the mechanism by moving the dial member to the dial position and dial the coupling and drive tube back to the initial zero position.

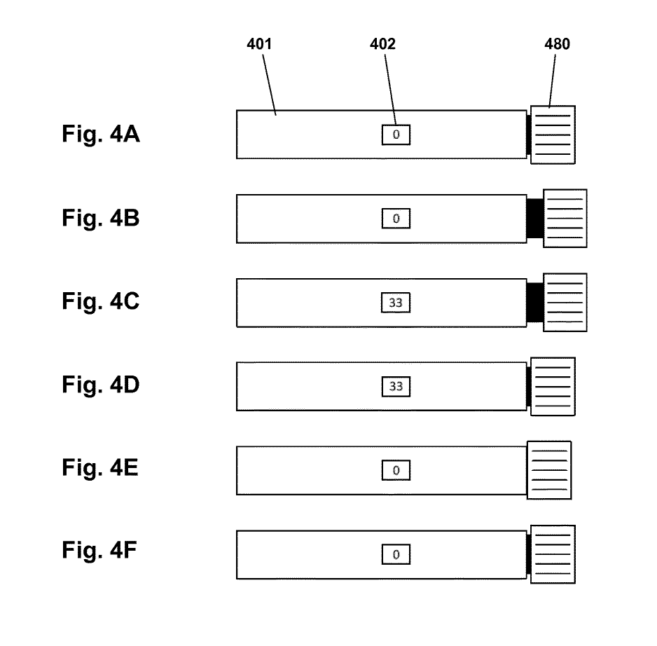

The above different modes and states are summarized in FIGS. 4A-4F showing how the user will experience the different modes and states.

FIG. 4A shows the proximal portion 401 of the pen-formed drug delivery device in the rest mode. In FIG. 4B the dial member 480 has been pulled out corresponding to the dial mode and in FIG. 4C a dose of 33 units of insulin (IU) showing in the display window 402 has been dialed. In FIG. 4D the dial has been pushed to the rest position whereby the set dose has been “parked”. In FIG. 4E the dial member has been pushed fully distally to the dosing mode and the set dose has been expelled, this being indicated by the scale drum having returned to the zero position. In FIG. 4F the pen is back in the rest mode.

Having regard to FIGS. 3A-3C an optional switch and sensor assembly was described, such an assembly being useful to control an electronic assembly adapted to detect a set and/or expelled dose. In the described embodiment no electronic circuitry is shown, however, it may be arranged inside the dial member as disclosed e.g. in WO 2014/128156 also disclosing a rotary sensor design which may be implemented in the sensor portion of the shown switch and sensor assembly, the document being incorporated by reference.

The provision of an axial mode switch in the above-described embodiment allows electronic circuitry to securely detect whether the rotary sensor rotates with the pen in the setting or dosing mode. Correspondingly, when the switch is in the on-state in the dial mode any rotational movement of the rotary sensor will be detected as setting a dose, whereas in the dosing mode the switch is in the off-state and any rotational movement of the rotary sensor will be detected as an expelled amount of drug. Having detected both a set dose and an expelled dose could be used in a check procedure in which a given dose is only registered when the detected expelled dose is identical to the detected set dose.

If the electronic circuitry is provided with a display, e.g. arranged corresponding to the dial member upper surface, the switch may also be used to control actuation of the display. For example, if the dial member is moved from the rest position to the dial position with no dose set the display may show the last detected dose together with time-since-last-dose information. The display may be turned off after e.g. 5 or 10 seconds or when the user starts to set a dose by turning the dial member.

The display can be configured to show data in different formats. For example, the display may be a two-line display in which time is shown using a HH:MM:SS stop watch design, this providing that the time since the last dose expelled from the device can be shown with a running second counter allowing a user to easily identify the shown information as a counting time value. After 24 hours the display may continue to display time in the HH:MM:SS format or change to a day and hour format.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device comprising or adapted to receive a cartridge with an axially displaceable piston, the drug delivery device comprising:
a housing comprising a distal end and a proximal end,
a piston rod adapted to engage and axially displace a piston in a loaded cartridge in a distal direction to thereby expel a dose of drug from the cartridge,
drive structure adapted to move the piston rod in the distal direction and comprising a drive spring,
a rotatable setting member allowing a user to set a dose amount to be expelled, the drive spring being strained during dose setting or being pre-strained, and
user actuated release structure for releasing the drive spring to thereby move the piston rod in the distal direction corresponding to the set dose,
wherein:
the setting member can be moved axially between an initial position and a proximal setting position, and between the initial position and a distal expelling position,
the setting member cannot be rotated to set a dose when in the initial position,
the setting member can be rotated to set a dose when in the proximal position, and
a strained drive spring is released when the setting member is moved axially from the initial position to the distal position.

2. A drug delivery device as in claim 1, wherein expelling of a set dose amount can be paused when the setting member is moved axially from the distal position to the initial position.

3. A drug delivery device as in claim 1 wherein the setting member is moved axially from the initial position to the distal position against a biasing force adapted to return the setting member to the initial position.

4. A drug delivery device as in claim 1, wherein the setting member is rotationally locked in the initial position.

5. A drug delivery device as in claim 1, further comprising a sensor system adapted to detect rotational movement during dose setting and dose expelling, the sensor system comprising electronic circuitry and a switch which is in:
a first state when the setting member is in the initial position,
a second state when the setting member in the proximal position, and
the first state when the setting member is in the distal position,
whereby a set dose is detected when the setting member is in the proximal position and an expelled dose is detected when the setting member is in the distal position.

6. A drug delivery device as in claim 5, the sensor system comprising a display adapted to display information relating to an expelled dose of drug.

7. A drug delivery device as in claim 6, wherein the display is turned on when the setting member is moved from the initial to the proximal position.

8. A drug delivery device as in claim 1, wherein the display is turned on when the setting member is moved from the distal to the initial position and an expelled dose has been detected.

9. A drug delivery device as in claim 8, wherein the display is turned on when a set dose has been fully expelled.

10. A drug delivery device as claim 1, wherein the electronic circuitry is coupled to the setting member and moves axially therewith.

11. A drug delivery device as in claim 10, wherein the setting member comprises an interior in which at least a portion of the electronic circuitry is arranged.

12. A drug delivery system comprising:
a drug delivery assembly,
a first setting member comprising coupling structure and electronic circuitry, and
a second setting member comprising coupling structure and no electronic circuitry,
the drug delivery assembly comprising:
a housing comprising a distal end and a proximal end,
a piston rod adapted to engage and axially displace a piston in a loaded cartridge in a distal direction to thereby expel a dose of drug from the cartridge,
an expelling mechanism adapted to move the piston rod in the distal direction and comprising a drive spring, the expelling mechanism being adapted to allow a user to (i) set a dose amount to be expelled, the drive spring being strained during dose setting or being pre-strained, and (ii) release the drive spring to thereby move the piston rod in the distal direction corresponding to the set dose,
coupling structure allowing either of the first and second setting members to be attached to the drug delivery assembly to form a drug delivery device,
wherein:
a setting member when attached can be moved axially between an initial position and a proximal setting position, and between the initial position and a distal position,
the setting member cannot be rotated to set a dose when in the initial position,
the setting member can be rotated to set a dose when in the proximal position, and
a strained drive spring is released when the setting member is moved axially from the initial position to the distal position.

13. A drug delivery system as in claim 12, wherein the electronic circuitry of the first setting member provides a sensor system adapted to detect rotational movement during dose setting and dose expelling.

14. A drug delivery system as in claim 13, wherein the sensor system comprises a switch which is in:
a first state when the setting member is in the initial position,
a second state when the setting member in the proximal position, and
the first state when the setting member is in the distal position,
whereby a set dose is detected when the setting member is in the proximal position and an expelled dose is detected when the setting member is in the distal position.
